(19) United States
(12) Patent Application Publication
(10) Pub. No.: US 2011/0257379 A1
(43) Pub. Date: Oct. 20, 2011

(54) LEAD COMPOUND OF ANTI-HYPERTENSIVE DRUG AND METHOD FOR SCREENING THE SAME

(76) Inventor: Hsin-Hsien Wu, Xindian City (TW)

(21) Appl. No.: 13/170,107

(22) Filed: Jun. 27, 2011

Related U.S. Application Data
(62) Division of application No. 12/039,713, filed on Feb. 28, 2008.

(51) Int. Cl. C07D 285/135; C07D 405/06; C07D 417/06; C07C 229/64; C07C 235/84; C07C 245/08; C07D 403/06

(52) U.S. Cl. 534/664; 534/851; 548/365.7; 548/455; 562/454; 560/19; 544/300; 548/187

(57) ABSTRACT

A system for screening a small molecule library with 250,000 molecules to find out a compound of an anti-hypertensive drug aiming at human Angiotensin II type IA receptor is provided. The system includes a first database having a three-dimensional structure datum of a human Angiotensin II type IA receptor, a second database having molecular data of a plurality of small molecules, and a computer acquiring the three-dimensional structure datum and the molecular data from the first database and the second database respectively, wherein the computer has a molecular docking software for calculating a free energy of the human Angiotensin II type IA receptor bound to each of the plurality of small molecules, ranks the plurality of small molecules according to the respective free energy so as to select a top small molecule in the ranking as the compound of the drug.

### CROSS-REFERENCE TO RELATED APPLICATIONS

This application is a division and claims the benefit of priority from U.S. patent application Ser. No. 12/039,713, filed on Feb. 28, 2008, which is now pending, the full disclosures of which are incorporated herein by reference.

### FIELD OF THE INVENTION

The present invention relates to a lead compound of a drug and a method for screening the lead compound, and more particularly to a lead compound of an anti-hypertensive drug and a method for screening the same.

### BACKGROUND OF THE INVENTION

RAA (Renin-Angiotensinogen-Angiotensin I-Angiotensin II) system plays a key role in human blood pressure homeostasis, wherein Angiotensin II protein is a major active substance which functions at most tissues and endothelial cells through Angiotensin II receptor and stimulates the contractions of arterioles so as to elevate blood pressure. Therefore, one class of the anti-hypertensive drugs is Angiotensin II Receptor Antagonist, which blocks the binding between the Angiotensin II protein and the Angiotensin II receptor to lower the blood pressure. Other classes of anti-hypertensive drugs include adrenergic inhibitors, vasodilators, Angiotensin converting enzyme (ACE) inhibitors, Ca²⁺ channel blockers, and diuretics.

The first step of the drug development is to find out a lead compound, which is a molecule that may affect a particular biological target in a desired manner; for instance, it may bind particularly well to a protein. A conventional technique of High Throughput Screening (HTS) is used to test a large number of molecules for biological activity. Highly automated liquid handling and detecting systems using robotic devices employed in the conventional HTS can only perform hundreds or thousands of screenings per day. Besides, the conventional HTS does not directly identify a drug. Namely, the conventional HTS cannot evaluate the bioavailability, pharmacokinetics, toxicity, or specificity of an active molecule. Further studies of the molecules identified by the conventional HTS are required to further identify a potential lead compound of a new drug.

In order to cut down the time and money spent on screening the lead compound, several new strategies have been developed to substitute the conventional HTS, where one is to simulate drug-receptor interactions using computational methods based on bioinformatics, the so-called “Virtual High-Throughput Screening (vHTS)”, which is also called “in-silico virtual screening” or “computer-based drug discovery”. With today's computational resources, millions of compounds can be screened in a few days. However, the prior anti-hypertensive drugs are only screened and developed through the conventional screening method, and there is no prior art using the vHTS method to screen a lead compound aimed at Angiotensin II type IA Receptor. There must be a lot of unscreened molecules that can be developed as new drugs.

Based on the above, it is necessary to provide a new method and system for screening a lead compound of a drug, especially an anti-hypertensive drug, to discover and develop new drugs.

### SUMMARY OF THE INVENTION

In accordance with one aspect of the present invention, a system for screening a compound of a drug for a disease is provided. The system includes a first database having a three-dimensional structure datum of a human Angiotensin II type IA receptor, a second database having molecular data of a plurality of small molecules, and a computer acquiring the three-dimensional structure datum and the molecular data from the first database and the second database respectively, and having a molecular docking software for calculating a free energy of the human Angiotensin II type IA receptor bound to each of the plurality of small molecules.

Preferably, the molecular docking software further ranks the plurality of small molecules according to the respective free energy and selects a top small molecule in the ranking as the compound of the drug for the disease.

Preferably, the plurality of small molecules are ranked according to an incremental order of the respective free energy.

Preferably, the compound of the drug for the disease is a lead compound.

Preferably, the disease is one selected from a group consisting of a hypertension, a hyperaldosteronism, a congestive heart failure, a surgically induced vascular smooth muscle proliferation, a cardiovascular disease, a stroke, a myocardial infarction, a renal disease, a hepatitis, a cancer, a glaucoma, and a combination thereof.

Preferably, a number of the plurality of small molecules is higher than 250,000.

Preferably, the molecular data includes a two-dimensional structure information of the plurality of small molecules, and the computer further comprises a converting program for converting the two-dimensional structure information of the plurality of small molecules into a three-dimensional structure information thereof.

Preferably, the molecule docking software is one selected from a group consisting of an Autodock®, a Dock®, a Glide®, a Flex®, a Gold®, and an ICM®.

Preferably, the computer is one selected from a group consisting of a personal computer, a workstation, a supercomputer, a computational grid, a minicomputer, a laptop computer, a tablet PC, a PDA, an embedded computer, a wearable computer and a combination thereof.

In accordance with another aspect of the present invention, a method for screening a lead compound of a drug for a disease is provided. The method includes steps of obtaining a three-dimensional structure datum of a human Angiotensin II type IA receptor, obtaining a three-dimensional structure information of a plurality of small molecules, calculating a free energy of the human Angiotensin II type IA receptor bound to each of the plurality of small molecules based on the three-dimensional structure datum and the molecular data; ranking the plurality of small molecules according to the respective free energy, and selecting a top small molecule in the ranking as the lead compound.

Preferably, the disease is one selected from a group consisting of a hypertension, a hyperaldosteronism, a congestive heart failure, a surgically induced vascular smooth muscle proliferation, a cardiovascular disease, a stroke, a myocardial infarction, a renal disease, a hepatitis, a cancer, a glaucoma, and a combination thereof.

Preferably, a number of the plurality of small molecules is higher than 250,000.

Preferably, the method further comprises a step of converting a two-dimensional structure information of the plurality of small molecules for obtaining the three-dimensional structure information thereof

Preferably, the steps of calculating the free energy, ranking the plurality of small molecules, and selecting the lead compound are processed by using a molecular docking software.

Preferably, the molecule docking software is one selected from a group consisting of an Autodock®, a Dock®, a Glide®, a Flex®, a Gold®, and a ICM®.

Preferably, the plurality of small molecules are ranked according to an incremental order of the respective free energy.

Preferably, the method is accomplished by using a computer selected from a group consisting of a personal computer, a workstation, a supercomputer, a computational grid, a minicomputer, a laptop computer, a tablet PC, a PDA, an embedded computer, a wearable computer and a combination thereof.

In accordance with a further aspect of the present invention, a lead compound of an anti-hypertensive drug, comprising at least one selected from a group consisting of

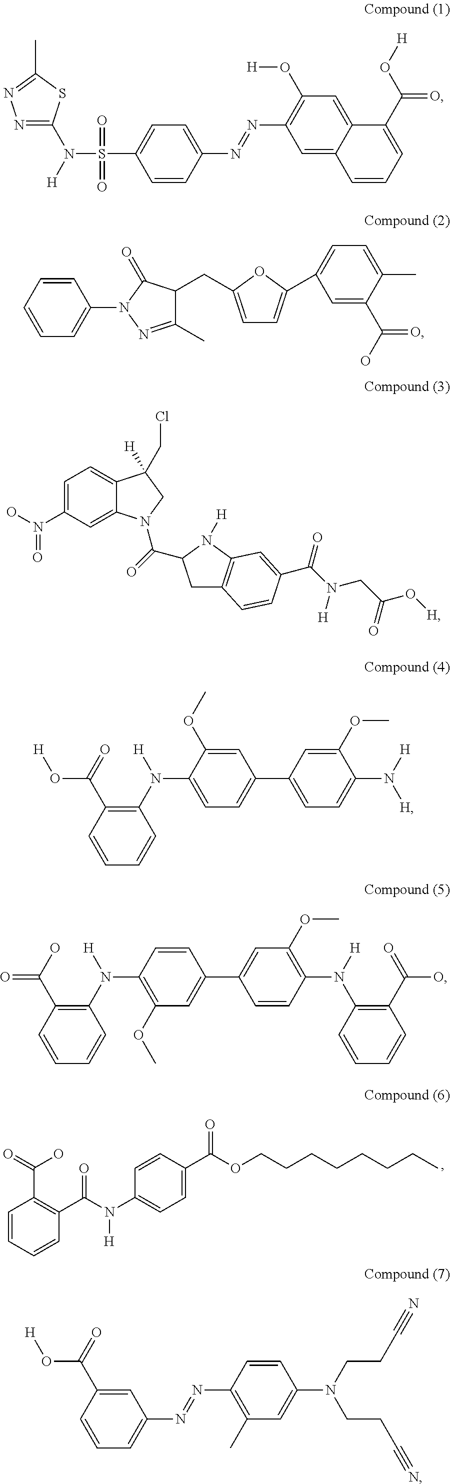

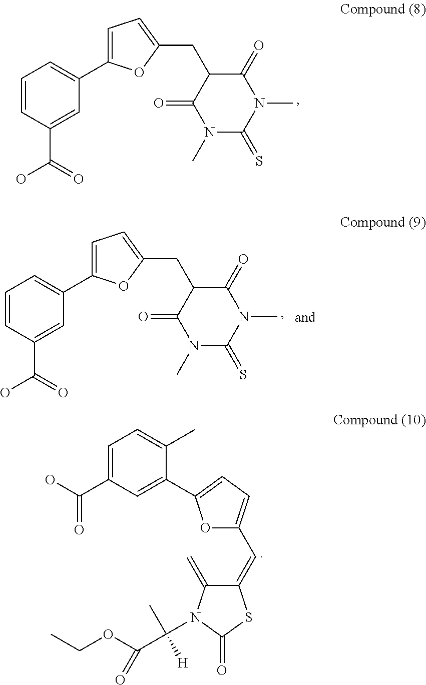

Additional objects and advantages of the invention will be set forth in the following descriptions with reference to the accompanying drawings, in which:

### BRIEF DESCRIPTION OF THE DRAWINGS

FIG. 1 is a schematic diagram showing the system for screening a lead compound of an anti-hypertensive drug according to a preferred embodiment of the present invention.

FIGS. 2(a)-2(e) show the molecular formulae of anti-hypertensive drugs in the prior art.

### DETAILED DESCRIPTION OF THE PREFERRED EMBODIMENT

The present invention will now be described more specifically with reference to the following embodiments. It is to be noted that the following descriptions of preferred embodiments of this invention are presented herein for the purposes of illustration and description only; it is not intended to be exhaustive or to be limited to the precise form disclosed.

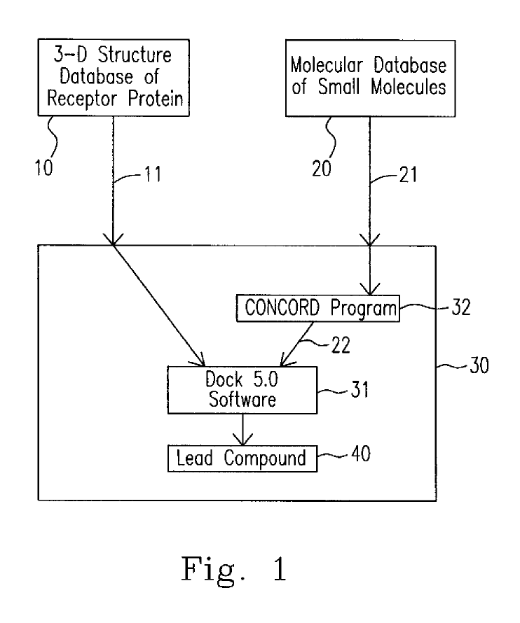

Please refer to FIG. 1, which is a schematic diagram showing the system for screening a compound of an anti-hypertensive drug according to a preferred embodiment of the present invention. The system includes a 3-D structure database 10 of receptor proteins, a molecular database 20 of small molecules, and a computer 30 having a molecular docking software 31, wherein the computer 30 acquires a three-dimensional structure datum 11 of the human Angiotensin II type IA receptor and molecular data 21 from the two above-mentioned databases 10, 20 respectively. If the molecular data 21 only includes two-dimensional structure information of the small molecules, the computer may further contain a converting program 32 for converting the two-dimensional structure information of the small molecules into three-dimensional structure information 22 thereof, wherein the converting program 32 used in the present invention is CONCORD® (Tripos Inc., St. Louis, Mo.). After processing the three-dimensional structure information 22 of the small molecules and the three-dimensional structure datum 11 of the human Angiotensin II type IA receptor, the molecular docking software 31 selects a top small molecule as a lead compound 40 of an anti-hypertensive drug. The criteria for the molecular docking software 31 to select the lead compound 40 may include the principles described as follows.

In the first step, the Lipinski (Pfizer) Rule of Five may be applied to estimate the potential for oral bioavailability. The rule states that, in general, an orally active drug has not more than 5 hydrogen bond donors (nitrogen or oxygen atoms with one or more hydrogen atoms), not more than 10 hydrogen bond acceptors, a molecular weight under 500 g/mol, and the lipophilicity with a partition coefficient log P less than 5. Lots of undesired molecules will be filtered out in this step.

After that, the free energy of the human Angiotensin II type IA receptor bound to each of the small molecules will be calculated by the molecular docking software 31, where the electrostatic interaction, van der Waals interaction, hydrogen bond interaction, and hydrophobic interaction therebetween will be taken into account. Each of the small molecules will be scored and ranked according to the free energy of binding with the human Angiotensin II type IA receptor, and the top ones can be selected as the lead compounds of the drug. The equation for calculating the free energy between two molecules can be expressed by:

$$E = {\sum }_{i = 1}^{liq} {\sum }_{j = 1}^{rec} [ \frac{{A}_{ij}}{{r}_{ij}^{a}} - \frac{{B}_{ij}}{{r}_{ij}^{b}} + 332 \times \frac{{q}_{i} \times {q}_{j}}{D \times {r}_{ij}} ] ,$$

wherein E is the interaction function between the two molecules; rᵢⱼ; is the distance between an atom i and an atom j; Aᵢⱼ is the parameter of van der Wall repulsion force; Bᵢⱼ; is the parameter of van der Waals attraction force; a is the number of van der Waals repulsion force power; b is the number of van der Waals attraction force power; qᵢ is the electric charge of atom i; qⱼ is the electric charge of the atom j; D is the dielectric function; 332 is the energy conversion parameter.

There are three major points for attention to achieve a successful virtual drug screening. First of all, the database of chemical molecules needs to be large enough. Secondly, the scoring function of the molecular docking software must be accurate, and the searching algorithm thereof must be efficient, wherein the searching algorithm can be selected from a flexible ligand docking algorithm, a “Place & Join” algorithm, an incremental algorithm, a genetic algorithm, an evolutionary algorithm, a de novo design algorithm, a rigid-body algorithm, a sequential growth algorithm, a hierarchical clustering algorithm, a k-means clustering algorithm, a partitioning clustering algorithm, a bi-clustering algorithm, or a CLICK algorithm. Thirdly, a fast processing speed must be supported by a powerful computer.

The most commonly used chemical databases in virtual screening include Available Chemical Directory (ACD) provided by the MDL®, the NCI Library provided by National Cancer Institute of United States, the World Drug Index (WDI) finished by Derwent, and so on. The above-mentioned databases provide more than 1 million of molecular data. In this preferred embodiment of the present invention, 250,000 molecules are collected from the above-mentioned databases for the virtual screening.

On the other hand, protein information can be obtained from the Protein Databank (http://www.pdb.org/). Because the actual 3-D structure of the human Angiotensin II type IA receptor Homology has not be discovered by X-ray diffraction or nuclear magnetic resonance (NMR) modeling, homology modeling is used to predict the 3-D structure thereof, wherein MODELLER© (maintained by Ben Webb at the Departments of Biopharmaceutical Sciences and Pharmaceutical Chemistry, and California Institute for Quantitative Biomedical Research, Mission Bay Byers Hall, University of California San Francisco) is a well-known tool in homology modeling, and the SWISS-MODEL Repository (http://swissmodel.expasy.org/repository) is the database of protein structures created with homology modeling.

Besides, the program, Dock® 5.0, available from University of California, San Francisco, Calif. is utilized to perform the calculation and the scoring function for selecting the lead compound 40 in accordance with the preferred embodiment of the present invention. Other well-known programs include AutoDock® (from Scripps Research Institute, La Jolla, Calif.), Ludi® (from Biosym Technologies, San Diego, Calif.), GOLD®, Glide® (from Schrödinger, LLC, New York, N.Y., USA), FlexX/FlexE/FlexS® (from Tripos Inc.), ICM® (from MolSoft), etc., and any of these scoring programs, either alone or a combination thereof, may be used for rescoring to obtain a more accurate result.

Furthermore, in a conventional virtual screening method, a workstation or a personal computer is employed as the hardware platform, where the drawback thereof is the slow processing speed. Other types of computers, such as a minicomputer, a laptop computer, a tablet PC, a PDA, an embedded computer, and a wearable computer, can be applied to the present invention. In this preferred embodiment of the present invention, a supercomputer with calculation speed of up to 10¹⁵-10¹⁸ times/sec at Computer Network Information Center of Chinese Academy of Sciences is provided to perform the molecular docking software 31, and the 250,000 small molecules in the molecular database 20 are screened and scored to find out the lead compound 40.

Finally, there are ten compounds selected as the lead compounds of anti-hypertensive drugs in the present invention, and the basic scaffolds thereof are illustrated as follows.

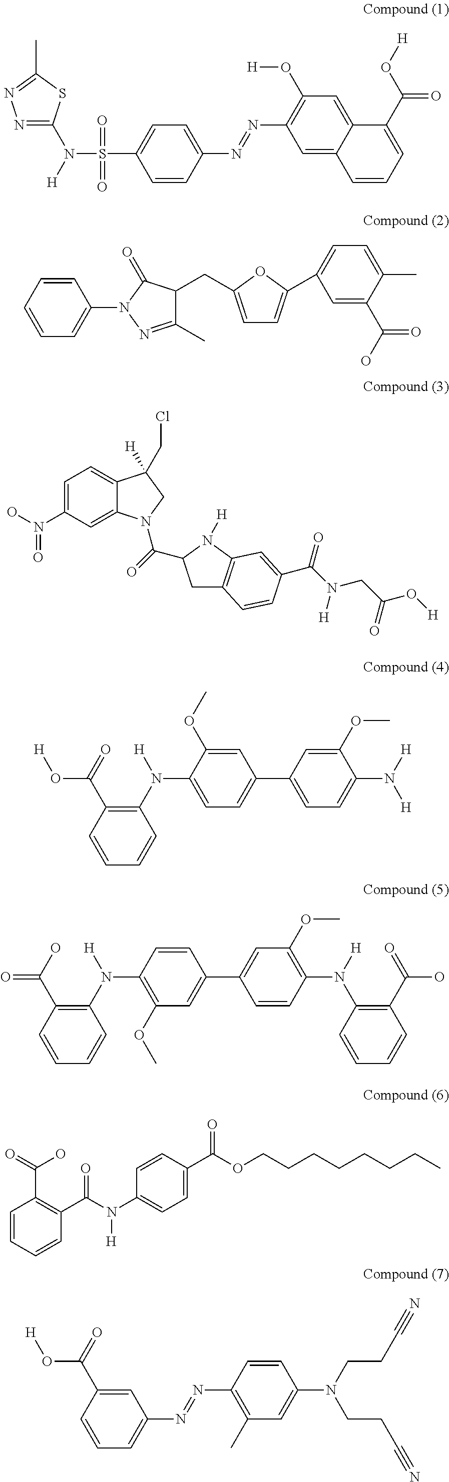

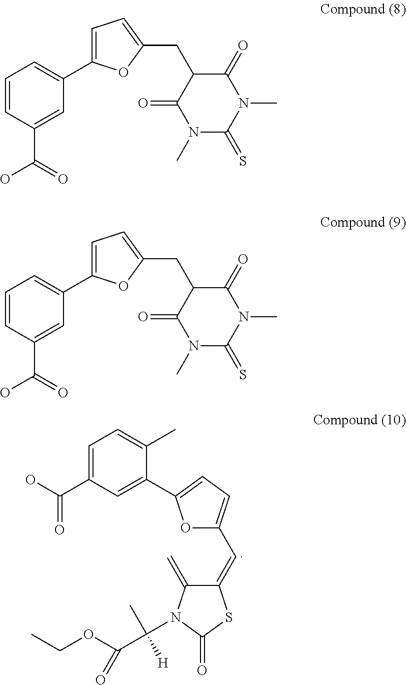

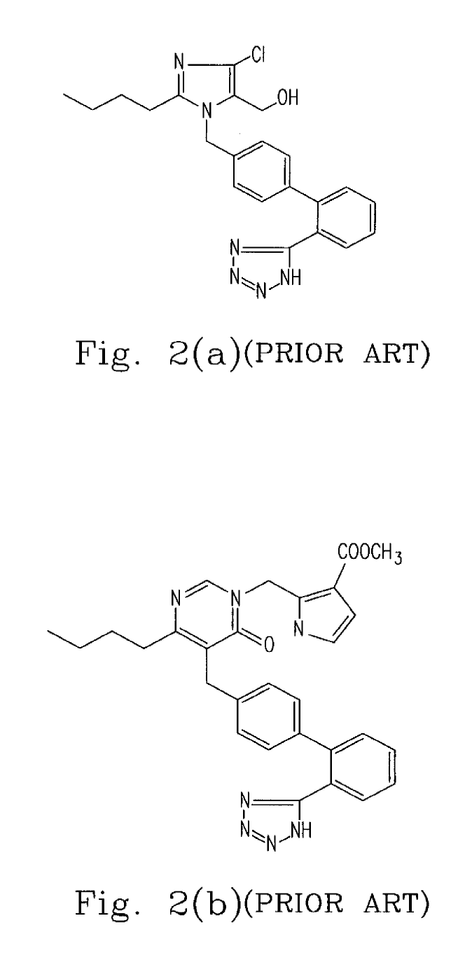

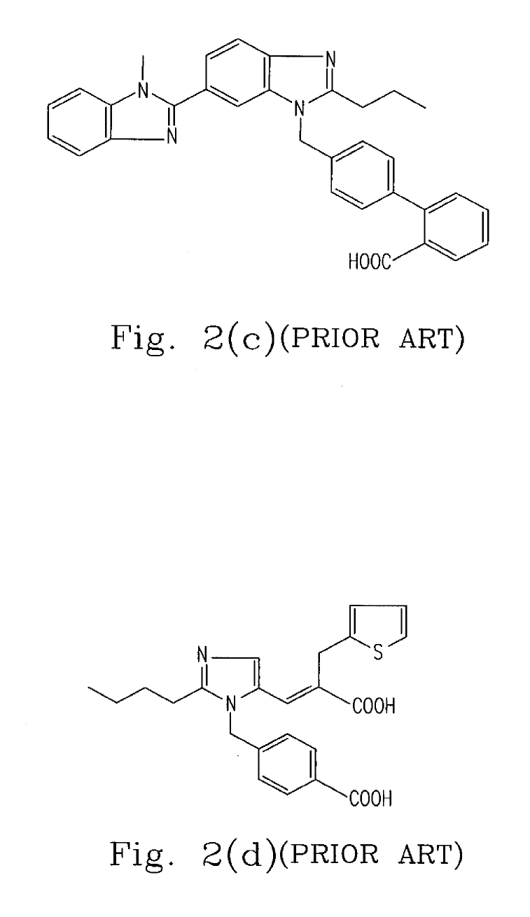

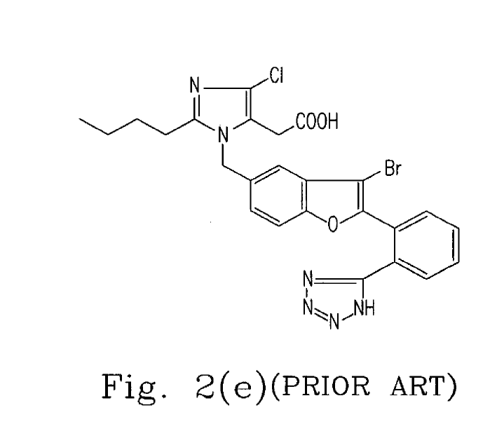

Please refer to FIGS. 2(a)-2(e), which illustrate the molecular formulae of anti-hypertensive drugs sharing similar tetrazole scaffolds in the state of the art, wherein the drug names of the formulae shown therein are Losartan, Milfasartan (LR-B/081), Telmisartan, Eprosartan, and Zolasartan, respectively. Compared with the scaffolds of the prior art, the scaffolds of these ten lead compounds provided by the present invention are apparently different, and these newly discovered lead compounds can provide potential new chemical entities (NCE) of anti-hypertensive drugs.

In conclusion, the present invention provides a system and a method for screening a lead compound of an anti-hypertensive drug, which combine the knowledge of cardiovascular receptorology, structural biophysics, bioinformatics, molecular pharmacology and computational molecular biology. In the prior art, the development of a new drug starts from experimental tests by using robotic devices to screen the potential lead compound of a drug, or by modifying substituent groups of the scaffolds of the existed drugs. Such a conventional screening method is time-consuming and expensive, and only modifying these old drugs can not greatly improve the curative effect thereof. The method and the system provided by the present invention use a powerful supercomputer and a well-known molecule docking software, Dock® 5.0, to implement an efficient virtual screening of the lead compound of a potential anti-hypertensive drug, and ten compounds which can be a new scaffold of a new drug are selected from 250,000 molecules.

Moreover, since the lead compound provided in the present invention could bind with the human Angiotensin II type IA receptor for lowering blood pressure, it could be used in other disease with a hypertensive symptom, such as hyperaldosteronism, congestive heart failure, surgically induced vascular smooth muscle proliferation, cardiovascular disease, stroke, myocardial infarction, renal disease, hepatitis, cancer, and glaucoma.

While the invention has been described in terms of what is presently considered to be the most practical and preferred embodiments, it is to be understood that the invention needs not be limited to the disclosed embodiments. On the contrary, it is intended to cover various modifications and similar arrangements included within the spirit and scope of the appended claims which are to be accorded with the broadest interpretation so as to encompass all such modifications and similar structures.

What is claimed is:

1. A lead compound of an anti-hypertensive drug, comprising at least one selected from a group consisting of 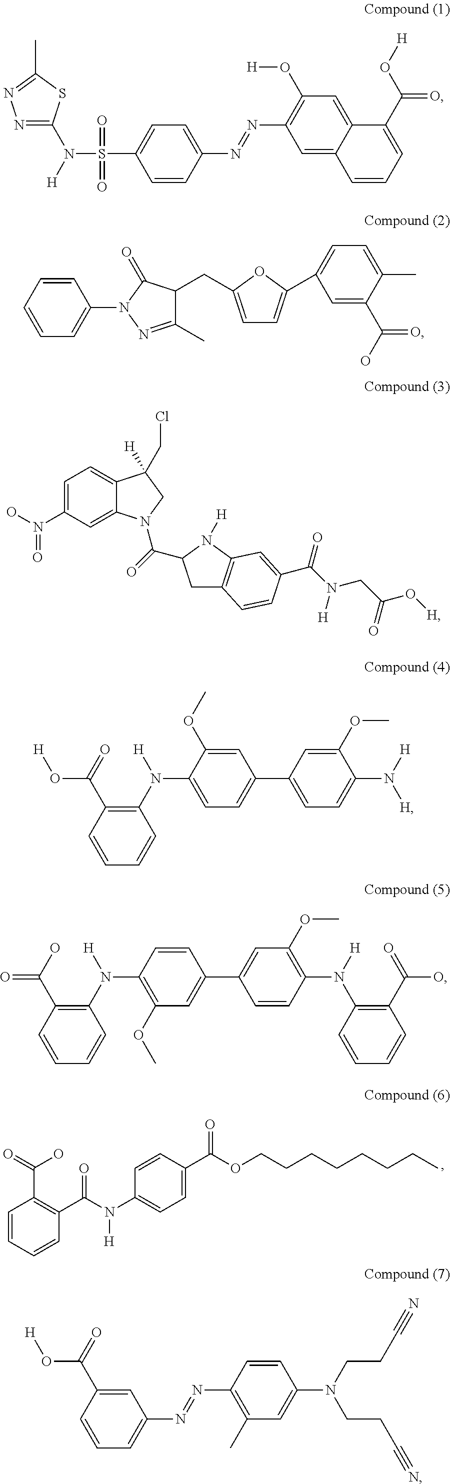 [chemical structure]
